(19) United States
(12) Patent Application Publication
(10) Pub. No.: US 2013/0261641 A1
(43) Pub. Date: Oct. 3, 2013

(54) INSERTS FOR DIVIDING A MEDICAL DEVICE LUMEN

(71) Applicant: BOSTON SCIENTIFIC SCIMED, INC., Maple Grove, MN (US)

(72) Inventors: Oscar R. CARRILLO, JR., Attleboro, MA (US); Samuel Raybin, Marlborough, MA (US); Paul Smith, Smithfield, RI (US); Tracy Andreotti, Milford, MA (US)

(73) Assignee: BOSTON SCIENTIFIC SCIMED, INC., Maple Grove, MN (US)

(21) Appl. No.: 13/791,211

(22) Filed: Mar. 8, 2013

Related U.S. Application Data
(60) Provisional application No. 61/618,258, filed on Mar. 30, 2012.

(51) Int. Cl. A61B 17/12; A61B 17/00

(52) U.S. Cl. CPC A61B 17/12013; A61B 17/00234
USPC 606/140; 606/1; 29/428

(57) ABSTRACT

An insert or elongate member divides the lumen of an elongated medical device, such as the working channel of an endoscope, into two or more secondary lumens. The insert or elongate member thereby improves the ability of the lumen of the elongated medical device to accommodate multiple items or uses. The insert or elongate member is capable of securing one item, such as a pull line, such that it does not interfere with another item positioned in the lumen of the elongated medical device or with another use of the lumen of the elongated medical device. In accordance with some embodiments, a ligating instrument comprises a ligating band dispenser, a pull line, and an elongate member having a dividing wall structure or a plurality of lumens, wherein the elongate member divides a working channel of an endoscope into a plurality of secondary lumens.

### CROSS REFERENCE TO RELATED APPLICATION

The present application claims priority to U.S. provisional application Ser. No. 61/618,258 filed Mar. 30, 2012, the disclosure of which is incorporated herein by reference in its entirety.

### TECHNICAL FIELD

The present invention relates to devices and methods for use with elongated medical devices, such as endoscopes and the like.

### BACKGROUND OF THE INVENTION

Various medical procedures involve the use of elongated medical devices having one or more lumens therethrough. For example, numerous medical procedures involve the use of endoscopes having one or more working channels.

One example of a medical procedure that can involve the use of an endoscope is a ligation procedure. Ligation is a medical procedure whereby a physician ties off or mechanically constricts a piece of body tissue with a ligature, such as a suture, clip or band. In certain procedures, the purpose of ligation may be to impede or obstruct the flow of blood, fluids and/or other bodily materials through the tissue. For example, a physician can remove target tissue by ligating it to obstruct circulation through the target tissue, thereby causing the tissue to die and slough off. The purpose of ligation also may be to hold tissue to be cauterized or resected, as in an endoscopic mucosal resection (EMR) procedure.

For ligating tissue inside a body cavity, orifice or lumen, physicians often use an endoscope to access the target tissue and ligate it. In one such form of endoscopic ligation, the physician attaches a ligation banding cap to the end of an endoscope. One or a plurality of ligation bands are stretched around the ligation banding cap and can be deployed by the physician. The physician uses the endoscope to position each stretched ligation band over the target tissue and then releases the band onto the tissue so that the band contracts and catches the tissue. The inward pressure of the ligation band constricts the target tissue.

Ligating instruments have been the subject of a number of patents and patent applications, including U.S. Pat. No. 5,259,366 to Reydel, et al.; U.S. Pat. No. 5,269,789 to Chin, et al.; U.S. Pat. No. 5,356,416 to Chu, et al.; U.S. Pat. No. 5,398,844 to Zaslaysky, et al.; U.S. Pat. No. 5,853,416 to Tolkoff; U.S. Pat. No. 5,857,585 to Tolkoff, et al.; U.S. Pat. No. 5,913,865 to Fortier, et al.; U.S. Pat. No. 5,968,056 to Chu, et al., U.S. Pat. No. 6,235,040 to Chu, et al.; U.S. Pat. No. RE 36,629 to Zaslaysky, et al., and U.S. Patent Application Publication No. 2008/0091218 to Richardson. The disclosures of these prior U.S. patents and patent application are expressly incorporated herein by reference.

A number of previous ligating instruments have relied upon deployment of the ligating bands by a pull line, for example a trigger wire, filament or string, that must be threaded through a lumen, in particular a working channel, of an endoscope. Although the wire, filament or string is usually much smaller in diameter or cross-section than the diameter of the working channel of the endoscope, the wire, filament or string can nevertheless hinder usage of the working channel for other uses or instruments due to the risk of obstruction and/or entanglement. The wire, filament or string may not lay flat against the inside wall of the working channel. For example, when the endoscope is in a tortuous configuration, the wire, filament or string can span the diameter of the working channel and thereby create an obstruction effectively larger than the cross-sectional area of the wire, filament or string.

There remains a need for improving the ability of a lumen of an elongated medical device, such as the working channel of an endoscope or other elongated medical device, to accommodate multiple items or uses, such as a wire or string and another item or use, without interference.

### SUMMARY OF THE INVENTION

The present disclosure relates to the use of one or more inserts or elongate members for improving the ability of a lumen of an elongated medical device, such as the working channel of an endoscope or other elongated medical device, to accommodate multiple items or uses, such as a pull line and another item or use, without interference.

In accordance with some embodiments, a medical device is provided comprising an elongate member for insertion in a lumen of an elongated medical device, such as the working channel of an endoscope or other elongated medical device. The elongate member is capable of securing one item, such as a pull line, such that it does not interfere with another item positioned in the lumen of the elongated medical device or with another use of the lumen of the elongated medical device.

In accordance with some embodiments, the insert or elongate member comprises a dividing wall structure, the dividing wall structure adapted to divide the lumen of the elongated medical device into a plurality of secondary lumens. A first secondary lumen may accommodate a pull line, and a second secondary lumen may accommodate a different instrument or another use, such as suction, flushing, etc.

In accordance with some embodiments, a ligating instrument for deploying one or more ligating bands inside the body comprises: (a) a ligating band dispenser comprising a support structure and at least one ligating band positioned on the support structure; and (b) a pull line extending from the ligating band dispenser and adapted to be pulled for deploying the ligating band(s) off of the support structure; wherein the ligating instrument further comprises an elongate member adapted to be positioned within a lumen of an elongated medical device; wherein the elongate member comprises a dividing wall structure, the dividing wall structure being adapted to divide the lumen of the elongated medical device into a plurality of secondary lumens; and wherein a first secondary lumen is adapted to accommodate the pull line.

In some embodiments, the ligating instrument further comprises an actuator member for pulling a proximal end of the pull line. The elongated medical device may be an endoscope. The ligating band dispenser may be adapted to be mounted on a distal tip of the endoscope. The actuator member may be adapted to be attached to a proximal end of the endoscope.

In accordance with some embodiments, a medical device comprises an elongate member adapted to be positioned within a lumen of an elongated medical device, wherein the elongate member comprises a dividing wall structure, the dividing wall structure being adapted to divide the lumen of the elongated medical device into a plurality of secondary lumens. The plurality of secondary lumens may comprise a first secondary lumen adapted to accommodate an actuation element. The medical device may comprise a ligating band dispenser comprising a support structure and at least one ligating band positioned on the support structure, wherein the ligating band dispenser is adapted to be mounted on a distal tip of the elongated medical device. The medical device may comprise an actuator member, wherein the actuator member is adapted to be attached to a proximal end of the elongated medical device. The actuation element may comprise, for example, one of a push element, a pull element, a torsion element, or an electrical actuation element. For example, the actuation element may comprise a pull line, for example a wire, filament or string.

In accordance with some embodiments, a method of assembling a first medical device to a second elongated medical device comprises inserting an elongate member of the first medical device into a lumen of the second elongated medical device, wherein the elongate member of the first medical device comprises a dividing wall structure, such that the step of inserting the elongate member of the first medical device into the lumen of the second elongated medical device separates the lumen of the second elongated medical device into a plurality of secondary lumens. The first medical device may comprise a ligating band dispenser comprising a support structure and at least one ligating band positioned on the support structure, and the method may comprise the step of mounting the ligating band dispenser on a distal tip of the second elongated medical device. The first medical device further may comprise an actuator member, and the method may comprise the step of attaching the actuator member to a proximal end of the second elongated medical device.

### BRIEF DESCRIPTION OF THE DRAWINGS

FIG. 1 is a schematic view of a prior art endoscopic working channel having a pull line extending therethrough.

FIG. 2 is an end view of an elongate member of a medical device in accordance with one embodiment of the invention.

FIG. 3 shows a medical device in accordance with one embodiment of the invention assembled to an endoscope.

FIG. 4 shows a cross-sectional view taken along line A-A of FIG. 3.

FIG. 5 shows a cross-sectional view of an elongate member of a medical device in accordance with another embodiment of the invention positioned in a lumen of an elongated medical device.

FIG. 6 shows a cross-sectional view of an elongate member of a medical device in accordance with another embodiment of the invention positioned in a lumen of an elongated medical device.

FIG. 7 shows a cross-sectional view of an elongate member of a medical device in accordance with another embodiment of the invention positioned in a lumen of an elongated medical device.

FIG. 8 shows a cross-sectional view of an elongate member of a medical device in accordance with another embodiment of the invention positioned in a lumen of an elongated medical device.

FIG. 9 shows a cross-sectional view of an elongate member of a medical device in accordance with another embodiment of the invention positioned in a lumen of an elongated medical device.

FIG. 10 shows a cross-sectional view of an elongate member of a medical device in accordance with another embodiment of the invention positioned in a lumen of an elongated medical device.

FIG. 11 shows a cross-sectional view of an elongate member of a medical device in accordance with another embodiment of the invention positioned in a lumen of an elongated medical device.

FIG. 12 shows a cross-sectional view of an elongate member of a medical device in accordance with another embodiment of the invention positioned in a lumen of an elongated medical device.

### DETAILED DESCRIPTION

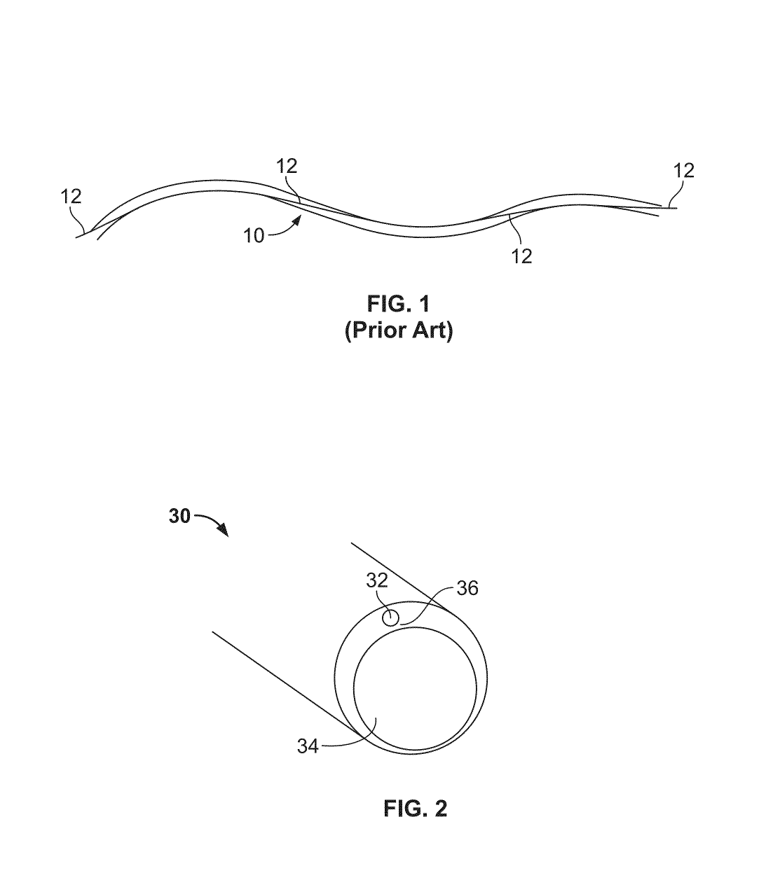

FIG. 1 is a schematic view of a prior art endoscopic working channel 10 having a pull line 12, such as a wire, filament or string, extending therethrough. Although the pull line 12 is much smaller in diameter or cross-section than the diameter of the working channel 10, the pull line 12 can hinder usage of the working channel 10 for other uses or instruments due to the risk of obstruction and/or entanglement. As shown in FIG. 1, when the endoscope is in a tortuous configuration, causing the working channel 10 to be in a tortuous configuration, the pull line 12 can span the diameter of the working channel 10, either partially or completely, and thereby create an obstruction effectively larger than the cross-sectional area of the pull line 12.

FIG. 2 is an end view of an elongate member 30 of a medical device in accordance with one embodiment of the invention. The elongate member 30 has a dividing wall structure 36 that results in plurality of lumens 32, 34. The first lumen 32 is adapted to accommodate an actuation element, such as a pull line. The second lumen 34 is a working lumen, adapted to accommodate a different medical instrument or another use, such as suction, flushing, etc.

In some embodiments, the diameter of the first lumen 32 is approximately the same as or slightly larger than the diameter or width of the actuation element to be accommodated therein. In this way, the working lumen 34 may be relatively large.

FIG. 3 shows a first medical device 28 in accordance with one embodiment of the invention assembled to a second elongated medical device such as an endoscope 14.

The medical device 28 is in the form of a ligating instrument for deploying one or more ligating bands 44 inside the body of a patient. The ligating instrument 28 comprises an elongate member 30 similar to that shown in FIG. 2. The elongate member 30 has a dividing wall structure 36 that results in a plurality of lumens, including a first lumen 32 adapted to accommodate a pull line 38 and a second lumen 34 which acts as a working lumen, adapted to accommodate a different medical instrument or another use, such as suction, flushing, etc. The elongate member 30 is adapted to be positioned within a lumen 20 of an elongated medical device, such as endoscope 14, in order to separate the lumen 20 of the elongated medical device into a plurality of secondary lumens 32, 34. In this embodiment, the elongate member 30 is a multi-lumen tubular insert.

The ligating instrument 28 of FIG. 3 comprises a ligating band dispenser 40 comprising a support structure 42 and at least one ligating band 44 positioned on the support structure 42. The ligating band dispenser 40 is adapted to be mounted on a distal tip 18 of the endoscope 14. The pull line 38 extends through the elongate member 38 to the ligating band dispenser 40 and is adapted to be pulled at its proximal end for deploying the ligating bands 44 sequentially off of the support structure 42.

The ligating instrument 28 further comprises an actuator member 50 for pulling the proximal end of the pull line 38. The actuator member 50 is adapted to be attached to a proximal end 16 of the endoscope 14. In a manner as is known in the art, the actuator member 50 can be actuated in steps in order to pull the pull line 38 in steps for deploying the ligating bands 44 sequentially off of the support structure 42. The actuator member 50 may be attached to the proximal end of the elongate member 30 in a manner that does not obstruct access to the second lumen 34 of the elongate member 30, in order that the second lumen 34 may adequately and reliably accommodate a different medical instrument or another use.

As can be appreciated from the above description, the use of one or more multiple-lumen tubular inserts or elongate members 30 improves the ability of a lumen of an elongated medical device, such as the working channel 20 of an endoscope 14 or other elongated medical device, to accommodate multiple items or uses, such as an actuation element 38 and another item or use, without interference. The tubular insert or elongate member 30 is capable of securing one item, such as a pull line 38, in a first secondary lumen 32 such that it does not interfere with another item positioned in the second secondary lumen 34 or with another use of the second secondary lumen 34.

Certain components of the medical device 28 can be pre-assembled together prior to assembling the medical device 28 to a second medical device such as endoscope 14. For example, in some embodiments, the elongate member 30 may be pre-assembled to the actuator member 50, with the actuation element 38 extending through a first lumen 32 of the elongate member 30. To assemble the medical device 28 to the endoscope 14, the elongate member 30 is inserted into the working channel 20 of the endoscope 14 at the proximal end 16 of the endoscope 14. Once the elongate member 30 is sufficiently inserted, the actuation element 38 is capable of being accessed at the distal end 18 of the endoscope 14. The actuation element 38 then may be attached to an actuation element associated with the ligating bands 44 (for example another pull line segment) by any suitable means, for example by hook and loop, adhesive, friction-fit type connections, knotting, etc.

In another example, in some embodiments, the elongate member 30 may be pre-assembled to the ligating band dispenser 40, with the actuation element 38 extending through a first lumen 32 of the elongate member 30. To assemble the medical device 28 to the endoscope 14, the elongate member 30 is inserted into the working channel 20 of the endoscope 14 at the distal end 18 of the endoscope 14. Once the elongate member 30 is sufficiently inserted, the actuation element 38 is capable of being accessed at the proximal end 16 of the endoscope 14. The actuation element 38 then may be attached to an actuation element associated with the actuator member 50 (for example, another pull line segment) by any suitable means, for example by hook and loop, adhesive, friction-fit type connections, knotting, etc.

In alternate embodiments, the medical device comprising an elongate member having a dividing wall structure for dividing the lumen of an elongated medical device into a plurality of secondary lumens may be used with any suitable elongated medical device having a working lumen. For example, the medical device comprising an elongate member having a suitable dividing wall structure may be used with endoscopes, cystoscopes, duodenal scopes, colonoscopes, guide tubes, introducers, catheters with lumens, and the like. When the elongate member is threaded or otherwise inserted into or positioned within the lumen of the elongated medical device, the elongate member separates the lumen of the elongated medical device into the plurality of secondary lumens.

One secondary lumen of the plurality of secondary lumens may comprise a first secondary lumen adapted to accommodate an actuation element, which is not limited to a pull line. The actuation element may be any suitable actuation element, for example a push element, a pull element, a torsion element, or an electrical actuation element. Another secondary lumen may accommodate other instruments or uses. The elongate member may divide the lumen of the elongated medical device into two, three or more secondary lumens.

The medical device comprising the elongate member having a dividing wall structure for dividing the lumen of an elongated medical device into a plurality of secondary lumens may be a ligating instrument or another suitable device. For example, the medical device may be forceps, a snare, a needle, a basket or any other suitable device.

The elongate member having a dividing wall structure for dividing the lumen of an elongated medical device into a plurality of secondary lumens may be formed by extrusion or any other suitable method of manufacture. For example, the elongate member may be made by molding. In some embodiments, the elongate member is made by extrusion out of a suitable plastic in order to provide the elongate member with sufficient flexibility for the desired application. In some embodiments, the elongate member and an actuation element such as pull line are extruded together, with the actuation element being made of a material that will not adhere to the elongate member, thereby allowing the actuation element to move relative to the elongate member. For example, the actuation element may be metal and the elongate member may be a suitable plastic material. The elongate member may be reinforced, for example by thickened wall portions, braids, coils, alternate materials overlaid, etc.

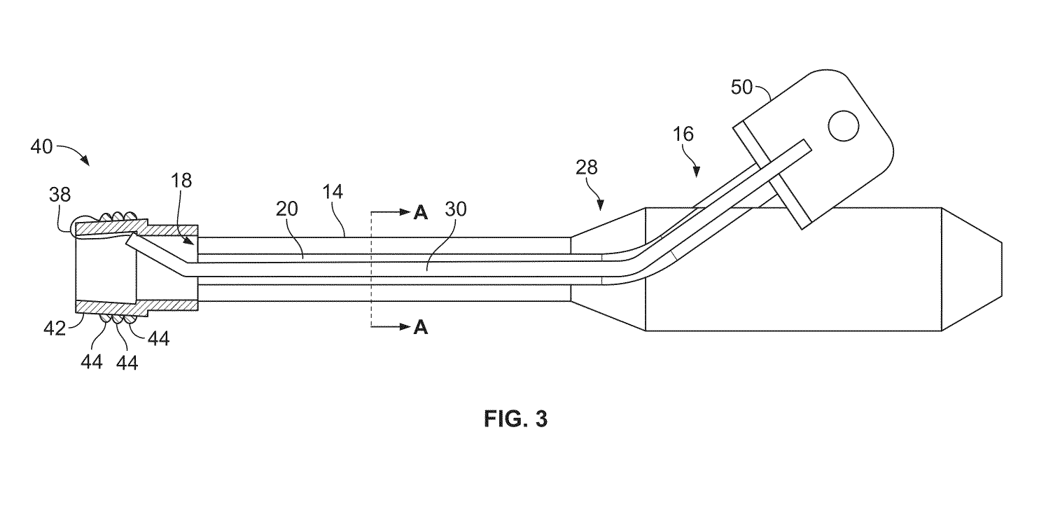

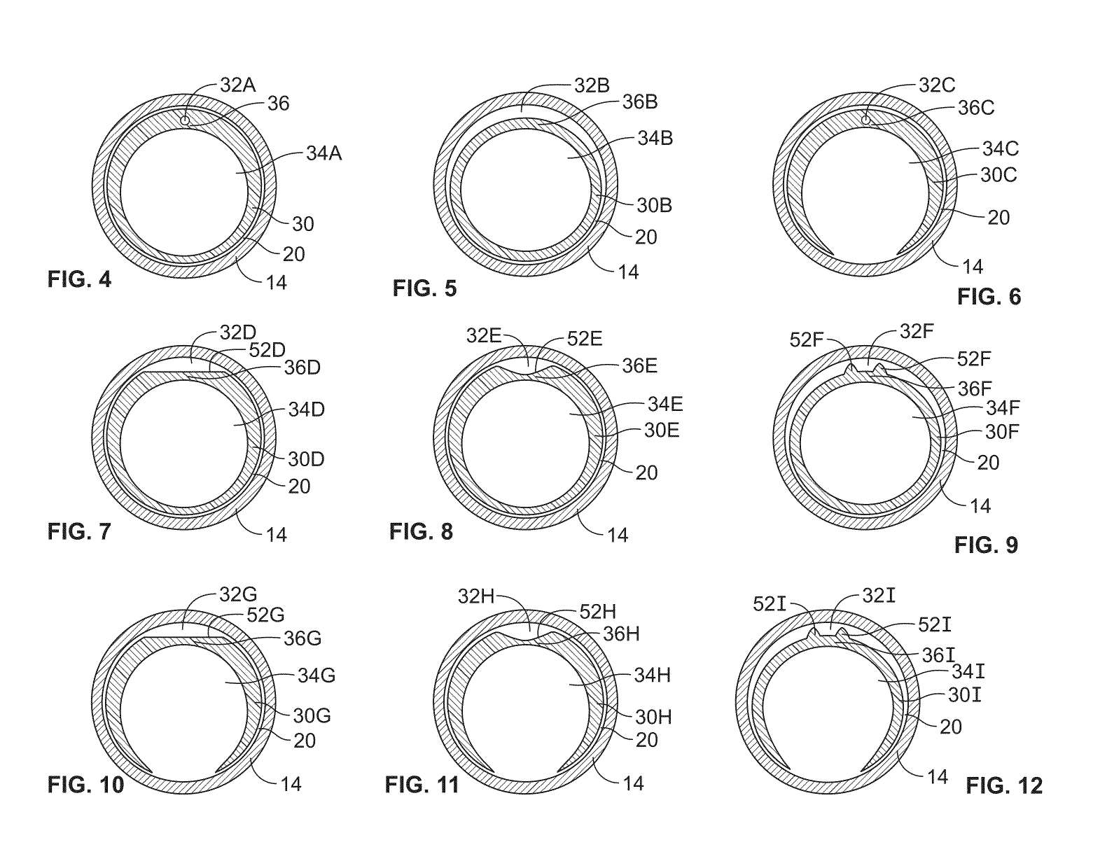

FIG. 4 shows a cross-sectional view taken along line A-A of FIG. 3. As can be seen in FIG. 4, when the elongate member 30 is positioned within a lumen 20 of an elongated medical device 14, the dividing wall structure 36 of the elongate member 30 divides the lumen 20 of the elongated medical device 14 into a plurality of secondary lumens 32A, 34A.

FIGS. 5-12 show cross-sectional views similar to that of FIG. 4, showing alternative embodiments of elongate members with dividing wall structures for dividing the lumen 20 of an elongated medical device 14 into a plurality of secondary lumens.

FIG. 5 shows an elongate member 30B positioned within a lumen 20 of an elongated medical device 14. In the embodiment of FIG. 5, when the elongate member 30B is positioned within the lumen 20 of the elongated medical device 14, some clearance space exists between the outer surface of the wall of the elongate member 30B and the inner surface of the lumen 20 of the elongated medical device 14. In one example, the cross-sectional shape of the elongate member 30B is circular, having an outer diameter smaller than the inner diameter of the lumen 20 of the elongated medical device 14, but other shapes leaving suitable clearance are of course possible. Thus, the dividing wall structure 36B of the elongate member 30B divides the lumen 20 of the elongated medical device 14 into a plurality of secondary lumens 32B, 34B, wherein the first secondary lumen 32B is created by the clearance space between the outer surface of the wall of the elongate member 30B and the inner surface of the lumen 20 of the elongated medical device 14. The first secondary lumen 32B may be adapted to accommodate an actuation element, such as a pull line.

FIG. 6 shows an elongate member 30C positioned within a lumen 20 of an elongated medical device 14. In the embodiment of FIG. 6, when the elongate member 30C is positioned within the lumen 20 of the elongated medical device 14, the dividing wall structure 36C of the elongate member 30C divides the lumen 20 of the elongated medical device 14 into a plurality of secondary lumens 32C, 34C. As can be seen in FIG. 6, the elongate member 30C does not by itself define two fully enclosed lumens. However, when the elongate member 30C is positioned inside the lumen 20 of the elongated medical device 14, the two secondary lumens 32C, 34C are defined. The second secondary lumen 34C is defined by two crescent-shaped arms of the elongate member 30C, which enable the elongate member to be securely positioned in the lumen 20. The embodiment of FIG. 6 may result in more space for the second secondary lumen 34C as compared to an embodiment in which the second secondary lumen is fully defined by the elongate member itself.

FIG. 7 shows an elongate member 30D positioned within a lumen 20 of an elongated medical device 14. In the embodiment of FIG. 7, when the elongate member 30D is positioned within the lumen 20 of the elongated medical device 14, the dividing wall structure 36D of the elongate member 30D divides the lumen 20 of the elongated medical device 14 into a plurality of secondary lumens 32D, 34D. As can be seen in FIG. 7, the elongate member 30D does not by itself define two fully enclosed lumens. However, when the elongate member 30D is positioned inside the lumen 20 of the elongated medical device 14, the two secondary lumens 32D, 34D are defined. The elongate member 30D has a flattened area 52D which leaves a clearance space between the outer surface of the wall of the elongate member 30D and the inner surface of the lumen 20 of the elongated medical device 14. The first secondary lumen 32D is created by this clearance space between the outer surface of the wall of the elongate member 30D and the inner surface of the lumen 20 of the elongated medical device 14.

FIG. 8 shows an elongate member 30E positioned within a lumen 20 of an elongated medical device 14. In the embodiment of FIG. 8, when the elongate member 30E is positioned within the lumen 20 of the elongated medical device 14, the dividing wall structure 36E of the elongate member 30E divides the lumen 20 of the elongated medical device 14 into a plurality of secondary lumens 32E, 34E. As can be seen in FIG. 8, the elongate member 30E does not by itself define two fully enclosed lumens. However, when the elongate member 30E is positioned inside the lumen 20 of the elongated medical device 14, the two secondary lumens 32E, 34E are defined. The elongate member 30E has a groove 52E which leaves a clearance space between the outer surface of the wall of the elongate member 30E and the inner surface of the lumen 20 of the elongated medical device 14. The first secondary lumen 32E is created by this clearance space between the outer surface of the wall of the elongate member 30D and the inner surface of the lumen 20 of the elongated medical device 14.

FIG. 9 shows an elongate member 30F positioned within a lumen 20 of an elongated medical device 14. In the embodiment of FIG. 9, when the elongate member 30F is positioned within the lumen 20 of the elongated medical device 14, the dividing wall structure 36F of the elongate member 30F divides the lumen 20 of the elongated medical device 14 into a plurality of secondary lumens 32F, 34F. As can be seen in FIG. 9, the elongate member 30F does not by itself define two fully enclosed lumens. However, when the elongate member 30F is positioned inside the lumen 20 of the elongated medical device 14, the two secondary lumens 32F, 34F are defined. The elongate member 30F has a two ridges 52F that define a channel between the outer surface of the wall of the elongate member 30F and the inner surface of the lumen 20 of the elongated medical device 14. The ridges 52F may be continuous or intermittent. The first secondary lumen 32F is created by the channel defined by the ridges 52F.

FIG. 10 shows an elongate member 30G positioned within a lumen 20 of an elongated medical device 14. In the embodiment of FIG. 10, when the elongate member 30G is positioned within the lumen 20 of the elongated medical device 14, the dividing wall structure 36G of the elongate member 30G divides the lumen 20 of the elongated medical device 14 into a plurality of secondary lumens 32G, 34G. As can be seen in FIG. 10, the elongate member 30G does not by itself define two fully enclosed lumens. However, when the elongate member 30G is positioned inside the lumen 20 of the elongated medical device 14, the two secondary lumens 32G, 34G are defined. The first secondary lumen 32G is defined by similar structure to that shown in FIG. 7, including a flattened area 52G, and the second secondary lumen 34G is defined by similar structure to that shown in FIG. 6.

FIG. 11 shows an elongate member 30H positioned within a lumen 20 of an elongated medical device 14. In the embodiment of FIG. 11, when the elongate member 30H is positioned within the lumen 20 of the elongated medical device 14, the dividing wall structure 36H of the elongate member 30H divides the lumen 20 of the elongated medical device 14 into a plurality of secondary lumens 32H, 34H. As can be seen in FIG. 11, the elongate member 30H does not by itself define two fully enclosed lumens. However, when the elongate member 30H is positioned inside the lumen 20 of the elongated medical device 14, the two secondary lumens 32H, 34H are defined. The first secondary lumen 32H is defined by similar structure to that shown in FIG. 8, including a groove 52H, and the second secondary lumen 34H is defined by similar structure to that shown in FIG. 6.

FIG. 12 shows an elongate member 301 positioned within a lumen 20 of an elongated medical device 14. In the embodiment of FIG. 12, when the elongate member 301 is positioned within the lumen 20 of the elongated medical device 14, the dividing wall structure 361 of the elongate member 301 divides the lumen 20 of the elongated medical device 14 into a plurality of secondary lumens 321, 341. As can be seen in FIG. 12, the elongate member 301 does not by itself define two fully enclosed lumens. However, when the elongate member 301 is positioned inside the lumen 20 of the elongated medical device 14, the two secondary lumens 321, 341 are defined. The first secondary lumen 321 is defined by similar structure to that shown in FIG. 9, including ridges 521, and the second secondary lumen 341 is defined by similar structure to that shown in FIG. 6.

The foregoing embodiments are merely examples of embodiments within the scope of the invention. Other embodiments are possible that incorporate one or more of the features or advantages of the above-described embodiments. This invention thus embraces other embodiments within the scope of the claims.

What is claimed is:

1. A medical device comprising:
an elongate member adapted to be positioned within a lumen of an elongated medical device;
wherein the elongate member comprises a dividing wall structure, the dividing wall structure being adapted to divide the lumen of the elongated medical device into a plurality of secondary lumens.

2. The medical device of claim 1, wherein the plurality of secondary lumens comprises a first secondary lumen adapted to accommodate an actuation element.

3. The medical device of claim 2, wherein the actuation element comprises a pull line.

4. The medical device of claim 2, wherein the actuation element comprises one of a push element, a pull element, a torsion element, or an electrical actuation element.

5. The medical device of claim 1, wherein the medical device further comprises a ligating band dispenser comprising a support structure and at least one ligating band positioned on the support structure, and wherein the ligating band dispenser is adapted to be mounted on a distal tip of the elongated medical device.

6. The medical device of claim 1, wherein the medical device further comprises an actuator member, and wherein the actuator member is adapted to be attached to a proximal end of the elongated medical device.

7. The medical device of claim 1, wherein the elongate member comprising the dividing wall structure is formed by extrusion.

8. A ligating instrument for deploying one or more ligating bands inside the body comprising:
(a) a ligating band dispenser comprising a support structure and at least one ligating band positioned on the support structure; and
(b) a pull line extending from the ligating band dispenser and adapted to be pulled for deploying said at least one ligating band off of the support structure;
wherein the ligating instrument further comprises an elongate member adapted to be positioned within a lumen of an elongated medical device;
wherein the elongate member comprises a dividing wall structure, the dividing wall structure being adapted to divide the lumen of the elongated medical device into a plurality of secondary lumens; and
wherein the plurality of secondary lumens comprises a first secondary lumen adapted to accommodate the pull line.

9. The ligating instrument of claim 8, further comprising an actuator member for pulling a proximal end of the pull line.

10. The ligating instrument of claim 8, wherein the elongated medical device is an endoscope.

11. The ligating instrument of claim 10, wherein the ligating band dispenser is adapted to be mounted on a distal tip of the endoscope.

12. The ligating instrument of claim 11, further comprising an actuator member for pulling a proximal end of the pull line, wherein the actuator member is adapted to be attached to a proximal end of the endoscope.

13. The ligating instrument of claim 8, wherein a plurality of ligating bands is positioned on the support structure of the ligating band dispenser, and wherein said ligating bands are adapted to be deployed sequentially by pulling the pull line.

14. A method of assembling a first medical device to a second elongated medical device comprising:
inserting an elongate member of the first medical device into a lumen of the second elongated medical device, wherein the elongate member of the first medical device comprises a dividing wall structure, the dividing wall structure being adapted to divide the lumen of the elongated medical device into a plurality of secondary lumens, such that the step of inserting the elongate member of the first medical device into the lumen of the second elongated medical device separates the lumen of the second elongated medical device into the plurality of secondary lumens.

15. The method of claim 14, wherein the first medical device further comprises a ligating band dispenser comprising a support structure and at least one ligating band positioned on the support structure, and further comprising the step of mounting the ligating band dispenser on a distal tip of the second elongated medical device.

16. The method of claim 14, wherein the first medical device further comprises an actuator member, and further comprising the step of attaching the actuator member to a proximal end of the second elongated medical device.

17. The method of claim 14, wherein the plurality of secondary lumens comprises a first secondary lumen adapted to accommodate an actuation element.

18. The method of claim 17, wherein the actuation element comprises a pull line.

19. The method of claim 18, wherein the pull line comprises a wire, filament or string.

20. The method of claim 17, wherein the actuation element comprises one of a push element, a pull element, a torsion element, or an electrical actuation element.
